(19) United States
(12) Patent Application Publication
(10) Pub. No.: US 2024/0227281 A1
(43) Pub. Date: Jul. 11, 2024

(54) MULTI-DIMENSIONAL BIOPRINTING SYSTEM AND METHOD

(71) Applicant: X Development LLC, Mountain View, CA (US)

(72) Inventor: Joseph Hollis Sargent, San Francisco, CA (US)

(21) Appl. No.: 18/151,003

(22) Filed: Jan. 6, 2023

(51) Int. Cl. B29C 64/106; B29C 64/209; B29C 64/255

(52) U.S. Cl. CPC B29C 64/106; B29C 64/209; B29C 64/255; B33Y 10/00

(57) ABSTRACT

The present disclosure relates to a container for receiving and supporting extruded biological material during a bioprinting process. The container comprises a reservoir configured to receive a suspension medium, a plurality of walls at least partially defining the reservoir, and a self-scaling port disposed in one or more of the plurality of walls.

### TECHNICAL FIELD

The present disclosure relates to multi-dimensional printing, and more particularly, to a multi-dimensional bioprinting system and method.

### BACKGROUND

Three-dimensional bioprinting is a process of creating cellular objects by depositing extrudable biological material, also referred to as bio-ink, in a three-dimensional space. Unlike some additive manufacturing techniques, three-dimensional bioprinting involves depositing cellular material in a printer bath (e.g., a petri dish) filled with a suspension material, such as a hydrogel, that allows for more precision and for arranging different cellular materials adjacent to one another to promote cellular growth. Current methods of bioprinting are time consuming, which is detrimental to the short-life of cells, and are also inefficient as the bio-ink must be switched to deposit a different cellular material. Typically, a bioprinter nozzle approaches from one axis (top-down) relative to the printing bath, and deposits material in a cross-sectional layer. To form a structure with two different cellular materials, the bio-ink must be changed (or a first extrusion tip must be removed and a second extrusion tip must be inserted for injecting a second bio-ink) before printing the next layer. While the bioprinter nozzle is not limited to printing from the bottom up, the current methods of bioprinting are limited to approaching the printer bath from one side (from the top) or a single axis.

### SUMMARY

A multi-dimensional bioprinting system and method disclosed herein allows for extrusion-based bioprinting from any side of a container containing a suspension medium. According to the method and configuration of the multi-dimensional bioprinting system, bioprinting three-dimensional cellular objects may reduce printing time and allow for multiple extrusions of bio-ink from multiple directions simultaneously. Additionally, the method and system of the present disclosure is more efficient for creating complex structures composed of multiple cellular materials.

In accordance with a first aspect, a container for receiving and supporting extruded biological material during a bioprinting process may include a reservoir configured to receive a suspension medium. A plurality of walls may at least partially define the reservoir, and a self-scaling port may be disposed in one or more of the plurality of walls.

In accordance with a second aspect, a container for receiving a printed biological material may include a reservoir configured to receive a suspension medium. A wall may at least partially define the reservoir, and a self-sealing portion may be integrated in the wall.

In accordance with a third aspect, a method of multi-dimensional bioprinting may include accessing a reservoir of a container through a pierceable portion of the container. The pierceable portion may be integrated into a wall of the container and the wall may at least partially define the reservoir. The method may include extruding a bio-ink into a suspension medium disposed in the reservoir.

In further accordance with any one or more of the foregoing first, second, and third aspects, a container for receiving a printed biological material and/or a method of multi-dimensional bioprinting may further include any one or more of the following aspects.

In one example, the reservoir may be enclosed by the plurality of walls.

In another example, an opening may be in communication with the reservoir.

In some examples, one or more of the plurality of walls may include a rigid material surrounding the self-scaling port or portion.

In other examples, the one or more of the plurality of walls may include a first self-scaling port and a second self-scaling port or portion spaced from the first self-sealing port.

In one example, the self-sealing port or portion may include a high-density foam.

In another example, the self-sealing port or portion may include a high-density rubber

In some examples, a frame may connect the one or more of the plurality of walls.

In another example, the frame may be configured for suspending the container.

In other examples, the plurality of walls may define a prism.

In one example, the container may include a second wall and a second self-scaling portion embedded in the second wall.

In one example, the second wall may at least partially define the reservoir.

In another example, a plurality of walls may define a cube.

In some examples, the container may include an opening in fluid communication with the reservoir.

In some other examples, the container may include a third wall and a third self-sealing portion embedded in the third wall.

In one example, the third wall may at least partially define the reservoir.

In one example, the method may include accessing the reservoir of the container through a second pierceable portion of the container.

In one example, the second pierceable portion may be integrated with a second wall of the container.

In another example, accessing the reservoir may include inserting a first extrusion needle through the pierceable portion of the wall and inserting a second extrusion needle through the second pierceable portion of the second wall.

In some examples, extruding a bio-ink may include extruding a first bio-ink using the first extrusion needle and extruding a second bio-ink using the second extrusion needle.

In some examples, the first bio-ink and the second bio-ink may have different cellular compositions.

### Definitions

As used herein, the term “about” means+/−10% of any recited value. As used herein, this term modifies any recited value, range of values, or endpoints of one or more ranges.

As used herein, the terms “top,” “bottom,” “upper,” “lower,” “above,” and “below” are used to provide a relative relationship between structures. The use of these terms does not indicate or require that a particular structure must be located at a particular location in the apparatus.

Some examples may be described using the expression “coupled” and “connected” along with their derivatives. For example, some arrangements may be described using the term “coupled” to indicate that two or more elements are in direct physical or electrical contact. The term “coupled,” however, may also mean that two or more elements are not in direct contact with each other, but yet still co-operate or interact with each other. The examples described herein are not limited in this context.

Other features and advantages of the present disclosure will be apparent from the following detailed description, figures, and claims.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a perspective view of a bioprinting system assembled in accordance with the teachings of the present disclosure;

FIG. 2 is a front view of the bioprinting system of FIG. 1;

FIG. 3 is a front, perspective view of an example container of the bioprinting system of FIG. 1;

FIG. 4 is a front view of a different example container for a bioprinting system assembled in accordance with the teachings of the present disclosure;

FIG. 5 is a front, perspective view of a different example container for a bio printing system assembled in accordance with the teachings of the present disclosure;

FIG. 6 is a front, perspective view of a different example container for a bio printing system assembled in accordance with the teachings of the present disclosure;

FIG. 7 is a front, perspective view of a different example container for a bio printing system assembled in accordance with the teachings of the present disclosure; and

FIG. 8 is a schematic diagram of a method of multi-dimensional bioprinting in accordance with the teachings of the present disclosure.

### DETAILED DESCRIPTION

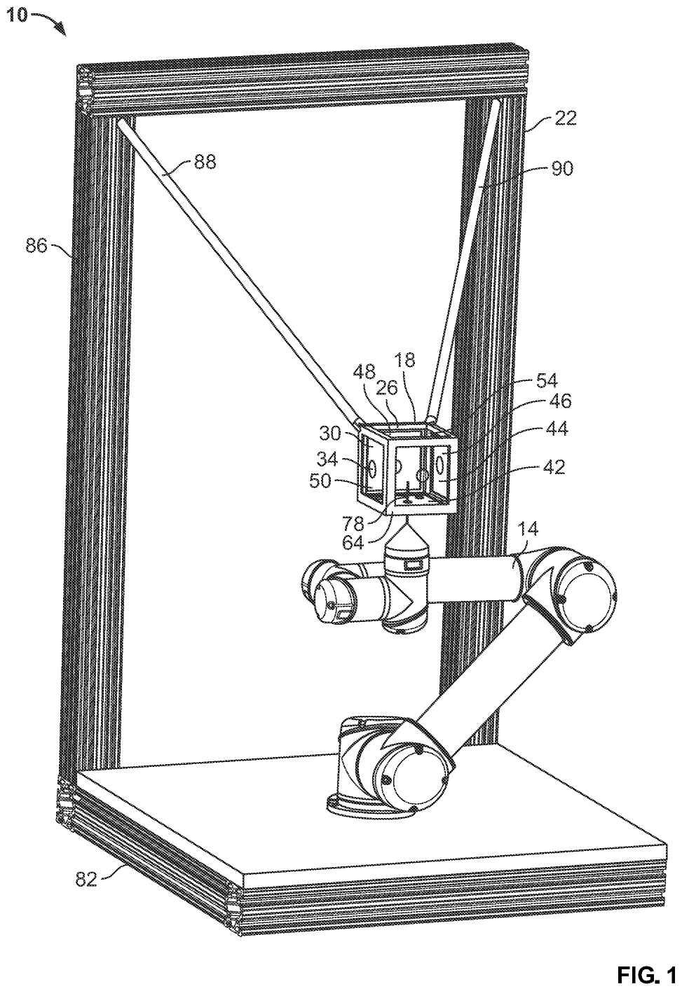

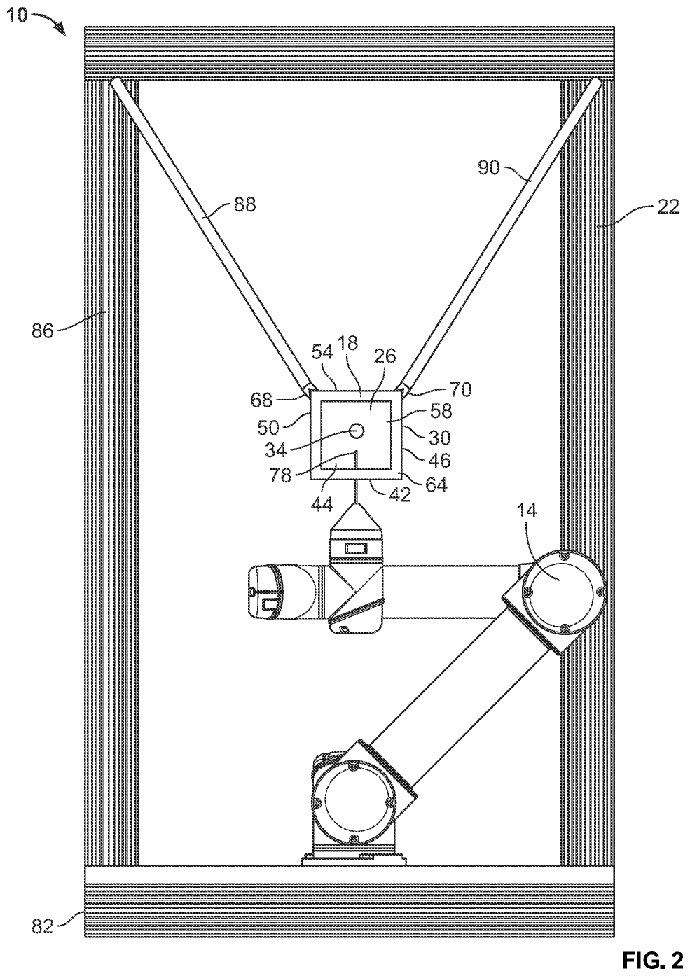

A multi-dimensional bioprinting system and method disclosed herein allows for extrusion-based bioprinting into a container and from any side of a container. In a first example, a multi-dimensional bioprinting system 10 in FIGS. 1 and 2 includes a bioprinter 14, a container 18, and a suspension structure 22, which suspends the container 18 above ground. In the illustrated example, the bioprinter 14 is disposed beneath the container 18 and the bioprinter accesses an interior volume 26 of the container 18 from below.

The container 18 is a printer bath and is configured to receive and support extruded or deposited biological material during a bioprinting process. The container 18 includes an interior volume or reservoir 26 configured to receive a suspension medium, a plurality of walls 30 at least partially defining the reservoir 26, and a self-sealing port 34 disposed in one or more of the plurality of walls 30. In FIGS. 1 and 2, an extrusion needle 38 of the bioprinter 14 is disposed through a self-sealing port 34 embedded in a bottom wall 42 of the multi-sided container 18. However, the bioprinter 14 may be arranged to print from any side of the container 18 through any of the plurality of walls 30.

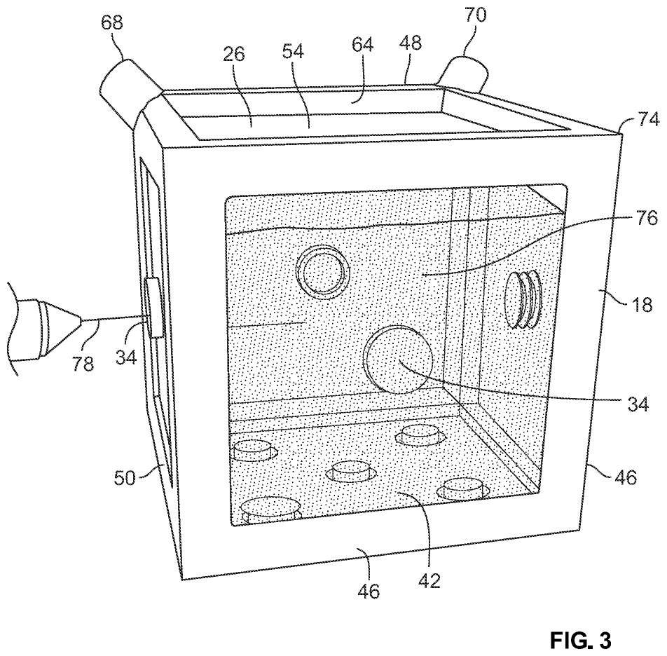

In FIG. 3, the container 18 is cubic and has first, second, third, and fourth sidewalls 44, 46, 48, 50, which are perpendicular relative to the bottom wall 42 and an opening 54 opposite the bottom wall 42. Each sidewall 44, 46, 48, 50 has a self-scaling port 34 centrally located and surrounded by a clear rigid material 58, together forming a leak-proof pierceable sidewall. The bottom wall 42 has five spaced out self-scaling ports 34 integrated with the clear rigid material 58 to form a leak-proof pierceable bottom wall 42. A cubic frame 64 reinforces the cubic container 18 and provides support arms 68, 70 at two of the eight corners 74 of the container 18. As shown in FIG. 3, the container 18 contains a suspension medium 76.

The rigid material 58 of the plurality of walls 30 of the container 18 may be selected from a range of different materials or combination of materials with varying visibility and material properties. In the illustrated example, the rigid material 58 is a plastic that may be shaped or molded according to the desired shape or number of self-scaling ports 34. The rigid material 58 is also clear to enhance visibility of the reservoir 26 of the container 18. However, in other examples, the rigid material 58 surrounding the self-scaling ports 34 may be opaque or tinted according to the sensitivities and requirements of the bio-ink. The rigid material 58 can be a metal, glass, ceramic, composite, or a combination of materials.

At each self-scaling port 34, an extrusion needle 78 may be inserted through the container wall 30 to deposit a bio-ink in the suspension medium. As shown in FIG. 3, an extrusion needle 78 from the bioprinter 14 is disposed through the self-sealing port 34 of one of the sidewalls 44, 46, 48, 50. The pliable material of the self-scaling port 34 may allow pivoting to reach a wide range of areas within the reservoir 26. Each of the plurality of walls 30 may have one or more self-scaling port 34, and the self-scaling port 34 may be any shape and size. In some examples, the self-sealing port 34 may occupy the majority surface area of the wall 30. The self-sealing port 34 may be high-density ethylene-vinyl acetate (EVA) foam, high-density rubber, or silicone with self-healing, self-scaling, and shape memory properties. The self-scaling port 34 may be pierced multiple times without failure and is configured to sealably close after being pierced.

Turning back to FIGS. 1-3, the frame 64 and suspension structure 22 are customized for a cubic container 18 to be suspended above ground. The suspension structure 22 includes a base board 82 upon which the bioprinter 14 sits, a back frame 86 perpendicular relative to the base board 82, and two brackets 88, 90 connecting the back frame 86 to the connecting arms 68, 70 of the frame 64 of the container 18. The frame 64 and suspension structure 22 may be arranged to suspend and support containers of different shapes and to work with bioprinters of various sizes. In the illustrated example, the bioprinter 14 is movable to access the container 18 from each side while the container 18 is held stationary. However, in other examples, the container 18 may be configured to rotate relative to the bioprinter 14. In these examples, the container 18 may be fixed to a rotatable mount coupled to the base 82 or the back frame 86.

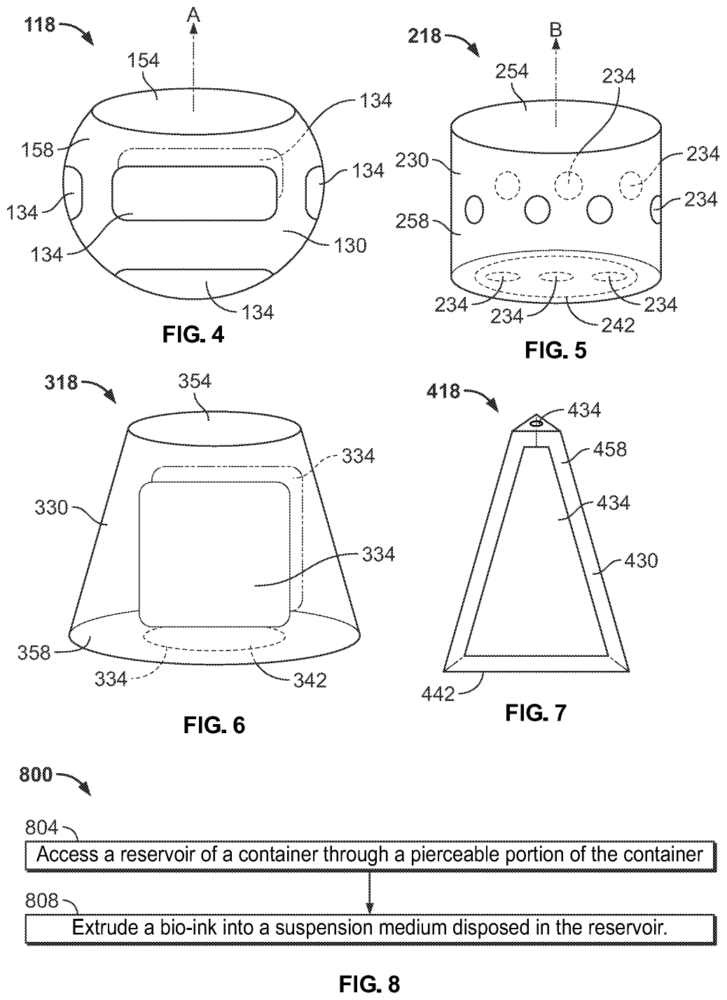

The container 18 may be any number of three-dimensional shapes. While the container 18 of FIGS. 1-3 is cubic, other example containers may be, for example, spherical, pyramidal, cylindrical, conical, frustoconical, triangular, cuboidal, hexagonal, or another prismatic shape. For example, in FIG. 4, a spherical container 118 includes a single spherical wall 130 and an opening 154 at a top portion of the container 118. A plurality of self-scaling ports 134 or portions are integrated with the wall 130 of the container 118 and are circumferentially spaced about a central axis A of the container 18. The self-scaling ports 134 are oblong and are spaced about 90 degrees relative to each other. However, in other examples, the self-sealing ports may be circular, rectangular, or other polygonal shape spaced about less than or more than 90 degrees relative to the other ports.

Other example containers have two or more walls and may be partially or completely enclosed (i.e., no opening to atmosphere). FIGS. 5-7 illustrate other example containers 218, 318, 418 that are different shapes. For example, in FIG. 5, a cylindrical container 218 includes a cylindrical wall 230, a bottom wall 242, and an opening 254 opposite the bottom wall 242. The cylindrical wall 230 is perpendicular relative to the bottom wall 242, and includes a plurality of circular, circumferentially disposed self-sealing ports 234. The bottom wall 242 includes a plurality of spaced apart self-scaling ports 234. In FIG. 6, the container 318 is frustoconical with an angled wall 330 that tapers toward an opening 354. The container 318 has a plurality of self-scaling portions 334 that span a large portion of the surface area. The self-scaling portion 334 of the bottom wall 342, for example, has a larger surface area than the rigid material 358 adjacent to the self-sealing portion 334. In some bioprinting applications, an anaerobic container may be desirable for printing biological material. In this case, the container may be completely enclosed on all sides, such as, for example, the container 418 of FIG. 7. In this example, the container 418 is prismatic with the majority of the surface area of the walls 430 being a self-scaling portion 434.

A suspension medium or material 76 of FIG. 3 is a synthetic or natural medium compatible with biological materials and supports bioprinted material in a three-dimensional space. The suspension material 76 may contain elements that support and promote the growth of the biomaterials. In some examples, the suspension material 76 may be a gel, such as a hydrogel, used for three-dimensional bioprinting applications. The suspension material 76 is self-healing such that the suspension material 76 allows for insertion of an injection needle 78, and then occupies the space of the needle when it is removed from the suspension material 76.

The term “bio-ink” as used herein may refer to any biological material suitable for bioprinting. For example, the material may be any biological material such as cells or biological polymers that can be printed using a printing device to create a biological structure.

FIG. 8 is a diagram of an example method 800 or process of multi-dimensionally bioprinting using a sealed, yet penetrable, container 18 containing a suspension medium 76. The method 800 may be performed using any of the containers 18 illustrated in FIGS. 1-3 or the containers illustrated in FIGS. 4-7. For case of reference, the method 800 is described with reference to the container 18 of FIGS. 1-3. Initially, the bioprinting process 800 may involve or use a computer, three-dimensional modeling software (e.g., Computer Aided Design, or CAD, software), bioprinter 14, and bio-ink. Once a CAD model is produced, the bioprinter 14 may read data from the CAD file and extrude or print various deposits bio-ink to fabricate a three-dimensional biological object according to the CAD file.

The method 800 includes a step 804 of accessing a reservoir 26 of a container 18 through a pierceable portion 34 of the container 18, such as through the self-scaling port 34 that is integrated into a wall 30 of the container 18, as shown in FIGS. 1-2. Accessing the reservoir includes inserting an extrusion needle 78 of a bioprinter 14 through the self-scaling port 34 so that the distal tip is disposed in the reservoir 26 of the container 18. Once the extrusion needle 78 is in a desired printing location, the method 800 includes a step 808 of extruding a bio-ink into the suspension medium 76 disposed in the reservoir 26 of the container 18. The method 800 may further include accessing the reservoir 26 of the container through a second pierceable portion of the container, such as through the self-sealing port 34 of the container 18 shown in FIG. 3.

In some examples, accessing the reservoir 26 includes inserting a first extrusion needle 78 through the pierceable portion 34 of the wall 30, and inserting a second extrusion needle 78 through the second pierceable portion 34 of the second wall. The step of extruding a bio-ink may include dispensing or extruding a first bio-ink through the first extrusion needle 78, and dispensing or extruding a second bio-ink using the second extrusion needle 78. The first and second extrusion needles 78 may be inserted simultaneously or consecutively, and the first bio-ink and the second bio-ink may have different cellular compositions. However, in other examples, the same extrusion needle 78 may be used to access the reservoir from different locations of the container 18 and for depositing different bio-inks. By accessing the reservoir 26 through different sides or walls 30 of the container 18, the biological object may be printed progressively according to a cellular structure, for example, rather than layer by layer from the bottom up.

Cell growth is directional and time dependent, and some biological structures require printing multiple types of cells in various arrangements and specific orders to encourage cellular bonding and growth. In one example method of the present disclosure, a bioprinter is programmed to print a cardiovascular system progressively by cellular structure. A first bio-ink composed of endothelial cells may be deposited first through an extrusion needle to form the inner lining of the vascular system. The entire endothelial structure may be printed, leaving voids for other cellular structures, before switching or using a second bio-ink. A second bio-ink composed of vascular cells may then be extruded into voids and/or around the endothelial structure. Because the bioprinter 14 can access the container 18 multi-dimensionally, the vascular cells of the second bio-ink may be placed adjacent to the endothelial cells in locations that could not be accessed from one side (i.e., the opening) of the container 18 without piercing the already printed endothelial structure. A third bio-ink composed of muscular tissue cells may then be printed into voids and/or around the vascular structure and/or endothelial structure.

The multi-dimensional bioprinting method 500 and containers 18, 118, 218, 318, 418 disclosed herein allow for efficient and logical multi-dimensional bioprinting, and may provide considerable benefits over current methods of three-dimensional bioprinting. The disclosed bioprinting containers 18, 118, 218, 318, 418 are simply constructed, and allows unconstrained multi-dimensional access for the bioprinter. Conventional methods of bioprinting require printing a biological structure from the bottom of the printer bath working up, printing in layers or cross-sections of the structure. When the biological structure includes more than one bio-ink per layer, each layer must be constructed at a time because forming or leaving voids and filling those voids in later around the first printed structure with a second bio-ink could not be done without piercing or rupturing the already printed tissue. Thus, printing layer-by-layer is arduous and time consuming, and often requires multiple printer nozzles or frequent changes of the bio-ink. By comparison, the bioprinting method of the present disclosure enables printing of an entire first structure of a first bio-ink before printing a second structure of a different bio-ink.

While this specification contains many specific implementation details, these should not be construed as limitations on the scope of any invention or of what may be claimed, but rather as descriptions of features that may be specific to particular examples of particular inventions. Certain features that are described in this specification in the context of separate examples can also be implemented in combination in a single example. Conversely, various features that are described in the context of a single example can also be implemented in multiple examples separately, or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the examples described herein should not be understood as requiring such separation in all examples, and it should be understood that the described program components and systems can generally be integrated together in a single product or packaged into multiple products.

Particular examples of the subject matter have been described. Other examples are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A container for receiving and supporting extruded biological material during a bioprinting process, the container comprising:
a reservoir configured to receive a suspension medium;
a plurality of walls at least partially defining the reservoir; and
a self-sealing port disposed in one or more of the plurality of walls.

2. The container of claim 1, wherein the reservoir is enclosed by the plurality of walls.

3. The container of claim 1, comprising an opening in communication with the reservoir.

4. The container of claim 1, wherein one or more of the plurality of walls comprises a rigid material surrounding the self-sealing port.

5. The container of claim 4, wherein the one or more of the plurality of walls comprises a first self-sealing port and a second self-sealing port spaced from the first self-sealing port.

6. The container of claim 1, wherein the self-sealing port comprises a high-density foam.

7. The container of claim 1, wherein the self-sealing port comprises a high-density rubber.

8. The container of claim 1, comprising a frame connecting the one or more of the plurality of walls, the frame configured for suspending the container.

9. The container of claim 1, wherein the plurality of walls define a prism.

10. A container for receiving a printed biological material, the container comprising:
a reservoir configured to receive a suspension medium;
a wall at least partially defining the reservoir; and
a self-sealing portion integrated in the wall.

11. The container of claim 10, comprising a second wall and a second self-sealing portion embedded in the second wall, the second wall at least partially defining the reservoir.

12. The container of claim 11, comprising a plurality of walls that define a cube.

13. The container of claim 11, comprising an opening in fluid communication with the reservoir.

14. The container of claim 13, comprising a third wall and a third self-sealing portion embedded in the third wall, the third wall at least partially defining the reservoir.

15. The container of claim 10, wherein the self-sealing portion is a high-density foam.

16. The container of claim 10, wherein the wall comprises a rigid material surrounding the self-sealing portion.

17-20. (canceled)

21. A container assembly for receiving extruded biological material during a bioprinting process, the container comprising:
a reservoir configured to receive a suspension medium;
a first wall and a second wall, the first wall and the second wall at least partially defining the reservoir;
a first self-sealing portion disposed in the first wall;
a second self-sealing portion disposed in the second wall; and
a frame connecting the first wall and the second wall, the frame configured for suspending the container.

22. The assembly of claim 21, wherein the self-sealing portion comprises a high-density foam.

23. The assembly of claim 21, wherein the self-sealing portion comprises a high-density rubber.

24. The assembly of claim 21, comprising a third wall, an opening opposite the third wall, and a plurality of self-sealing portions disposed in the third wall.
